(19) United States
(12) Patent Application Publication
(10) Pub. No.: US 2015/0283348 A1
(43) Pub. Date: Oct. 8, 2015

(54) HEADGEAR AND PATIENT INTERFACE DEVICE EMPLOYING SAME

(71) Applicant: KONINKLIJKE PHILIPS N.V., EINDHOVEN (NL)

(72) Inventors: Maureen Harp, Freedom, PA (US); Lauren Patricia Chodkowski, Pittsburgh, PA (US); Kevin Daniel Himes, Irwin, PA (US)

(21) Appl. No.: 14/402,183

(22) PCT Filed: May 22, 2013
(86) PCT No.: PCT/IB2013/054227

§ 371 Date: Nov. 19, 2014

Related U.S. Application Data
(60) Provisional application No. 61/650,511, filed on May 23, 2012.

(51) Int. Cl. A61M 16/06; A61M 16/00

(52) U.S. Cl. CPC A61M 16/0683; A61M 16/0057; A61M 2205/0216

(57) ABSTRACT

An elastic headgear that automatically adjusts to fit patients of different sizes and shape and that may be customized through selective placement of various accessories. The headgear includes a strap member having a central panel and a first arm member and a second arm extending from the central panel. The arm members each have at least one first attachment feature. The headgear also includes at least one accessory member having at least one second attachment feature, wherein the at least one second attachment feature is selectively mateable with the at least one first attachment feature to allow the at least one accessory member to be selectively attached to and positioned along either the first arm member or the second arm member at a plurality of different locations.

### CROSS-REFERENCE TO RELATED APPLICATIONS

This patent application claims the priority benefit under 35 U.S.C. §119(e) of U.S. Provisional Application No. 61/650,511, filed on May 23, 2012, the contents of which are herein incorporated by reference.

### BACKGROUND OF THE INVENTION

1. Field of the Invention

The present invention pertains to patient interface devices structured to deliver a flow of breathing gas to a user, and, in particular, to a headgear for a patient interface device.

2. Description of the Related Art

There are numerous situations where it is necessary or desirable to deliver a flow of breathing gas non-invasively to the airway of a patient, i.e., without intubating the patient or surgically inserting a tracheal tube into the patient's esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible sealing cushion on the face of a patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The sealing cushion typically has a support portion coupled to a sealing flap portion, which may integrated together as a single part or that may be separate components that when combined together in the final assembly provide the sealing and support functions. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

In designing patient interface devices, particularly those for pressure support therapy for treating OSA, accurate fit to a given patient is a critical measure of therapy success. Poor fit to a given patient can result in excessive mask leak (reduced therapy pressures), mask over tightening, development of sore, abrasions and/or indentations on the patient's face. Specifically, poor fit of headgear impacts therapy success in that comfort and seal are compromised.

### SUMMARY OF THE INVENTION

Accordingly, it is an object of the present invention to provide a headgear for a patient interface device that overcomes the shortcomings of conventional headgear. This object is achieved according to the present invention by providing an elastic headgear that automatically adjusts to fit patients of different sizes and shape and that may be customized through selective placement of various accessories.

In one embodiment, a headgear assembly for a patient interface device structured to deliver a flow of breathing gas to a patient is provided. The headgear assembly includes a strap member having a central panel and at least a first arm member extending from a first side of the central panel and a second arm member extending from a second side of the central panel opposite the first side, the central panel, the first arm member and the second arm member being made of an elastic non-textile material having at least one of a tensile modulus of 1.5 Mpa to 10 Mpa and an elongation without break of 400% to 1100%. In addition, the first arm member and the second arm member each have at least one first attachment feature. The headgear assembly also includes at least one accessory member having at least one second attachment feature, wherein the at least one second attachment feature is selectively mateable with the at least one first attachment feature to allow the at least one accessory member to be selectively attached to and positioned along either the first arm member or the second arm member at a plurality of different locations.

In another embodiment, a headgear assembly for a patient interface device structured to deliver a flow of breathing gas to a patient is provided. The headgear assembly includes a strap member including: (i) a base member made of an elastic material having at least one of a tensile modulus of 1.5 Mpa to 10 Mpa and an elongation without break of 400% to 1100%, wherein the base member includes a central panel portion and at least a first base arm member extending from a first side of the central panel portion and a second base arm member extending from a second, opposite side of the central panel portion, wherein the first base arm member and the second base arm member each have at least one first attachment feature, (ii) a central reinforcing portion coupled to and covering only a portion of the central panel portion, (iii) a first arm reinforcing portion coupled to and covering only a portion of the first base arm member, and (iv) a second arm reinforcing portion coupled to and covering only a portion of the second base arm member.

The central reinforcing portion, the first arm reinforcing portion and the second arm reinforcing portion are made of an inelastic material having at least one of a tensile modulus greater than or equal to 15 Mpa and an elongation without break of less than or equal to 60%. The headgear assembly also includes at least one accessory member having at least one second attachment feature (42, 76), wherein the at least one second attachment feature is selectively mateable with the at least one first attachment feature to allow the at least one accessory member to be selectively attached to and positioned along either the first base arm member or the second base arm member at a plurality of different locations.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the invention;

FIG. 2 is a top plan view of a strap member forming a part of a patient interface device of the system of FIG. 1 according to an exemplary embodiment of the present invention;

FIGS. 3 and 4 are side elevational views of pad members that may be selectively coupled to the strap member of FIG. 2;

FIG. 5 is a side elevational view of a tube clip that may be selectively coupled to the strap member of FIG. 2;

FIG. 6 is a top plan view of a strap member according to an alternative exemplary embodiment;

FIG. 7 is a schematic diagram of a system adapted to provide a regimen of respiratory therapy to a patient that employs the strap member of FIG. 6;

FIGS. 8 and 9 are schematic diagrams of systems adapted to provide a regimen of respiratory therapy to a patient according to alternative exemplary embodiments of the invention;

FIG. 10 is a bottom plan view of a strap member according to another alternative exemplary embodiment;

FIG. 12 is a side elevational view and FIG. 13 is a cross-sectional view taken along lines B-B of FIG. 11 of a pad members that may be selectively coupled to the strap member of FIG. 10;

FIG. 14 is a bottom plan view of a strap member according to still another alternative exemplary embodiment;

FIG. 15 is a top plan view of a strap member according to yet another alternative exemplary embodiment;

FIG. 16 is a top plan view of a base member forming a part of the strap member of FIG. 15;

FIG. 17 is a top plan view showing certain reinforcing members forming a part of the strap member of FIG. 15;

FIGS. 18, 19 and 20 are cross-sectional views taken along lines C-C, D-D, and E-E, respectively, of FIG. 15;

FIGS. 21 and 22 are schematic diagrams of systems adapted to provide a regimen of respiratory therapy to a patient that employ the strap member of FIG. 15;

FIGS. 23, 24 and 25 are top plan views of a strap member according to yet another alternative exemplary embodiment;

FIG. 26 is a bottom plan view of a base member forming a part of the strap member of FIG. 25; and

FIG. 27 is a cross-sectional view taken along lines F-E of FIG. 25.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

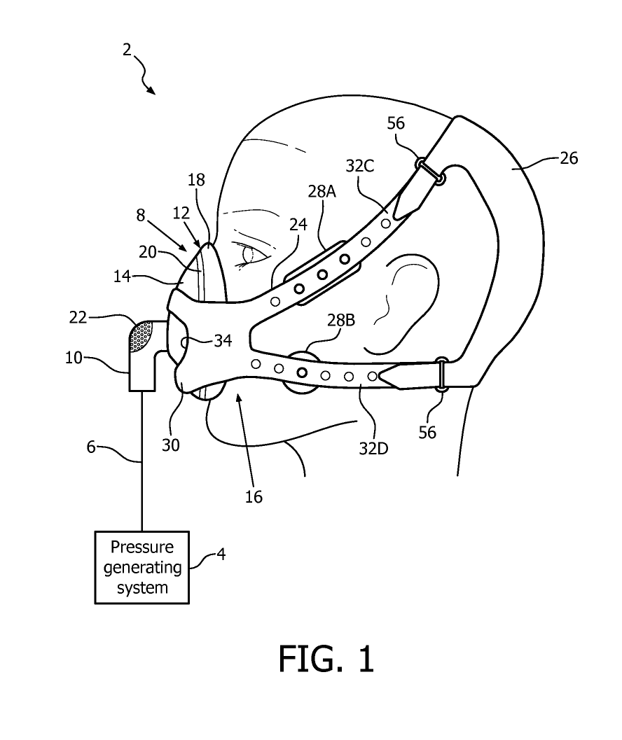

A system 2 adapted to provide a regimen of respiratory therapy to a patient according to one exemplary embodiment of the invention is generally shown in FIG. 1. System 2 includes a pressure generating device 4, a delivery conduit 6, and a patient interface device 8 including an elbow conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8.

In the illustrated embodiment, patient interface device 8 comprises a nasal/oral mask structured to cover the nose and mouth of the patient. However, other types of patient interface devices 8, such as, without limitation, a nasal mask that covers the patient's nose, or a full face mask that covers the patient's face, which facilitates the delivery of the flow of breathing gas to, and the removal of a flow of exhalation gas from, the airway of a patient may be used while remaining within the scope of the present invention.

In the embodiment shown in FIG. 1, patient interface device 8 includes a cushion assembly 12, a faceplate member 14 (also called a shell), and a headgear assembly 16. Cushion assembly 12 includes a cushion member 18 coupled to a support ring 20. In the exemplary embodiment, cushion member 18 is defined from a unitary piece of soft, flexible, cushiony, elastomeric material, such as, without limitation, silicone or an appropriately soft thermoplastic elastomer, or any combination of such materials. In addition, support ring 20 is made from a rigid or semi-rigid material, such as, without limitation, an injection molded thermoplastic or silicone, and facilitates secure fluid connection of cushion assembly 12 to faceplate member 14. Faceplate member 14 is also made of a rigid or semi-rigid material, such as, without limitation, an injection molded thermoplastic or silicone. An opening in faceplate member 14 to which elbow conduit 10 is coupled allows the flow of breathing gas from pressure generating device 4 to be communicated to an interior space defined by faceplate member 14 and cushion assembly 12, and then, to the airway of a patient. The opening in faceplate member 14 also allows the flow of exhalation gas (from the airway of such a patient) to be communicated to exhaust vent 22 provided in elbow conduit 10. As described in greater detail below, headgear assembly 16 is structured to enable patient interface device to be secured to the head of the patient in a manner that creates a suitable seal with cushion assembly 12.

In the exemplary embodiment, headgear assembly 16 includes strap member 24 that is adjustably coupled to a back piece 26. In addition, headgear assembly 16 further includes a number of pad members 28 (labeled 28A, 28B in the illustrated embodiment) that are able to be selectively coupled to strap member 24 at desired locations as described herein. Pad members 28 are each structured to provide one or more of a support, anti-slip and/or anti-red-mark (i.e., anti-pressure) functionality for patient interface device 8. In addition, as described elsewhere herein, other types of accessories, such as, without limitation, a tube positioning/management clip, are also able to be selectively coupled to strap member 24 at desired locations.

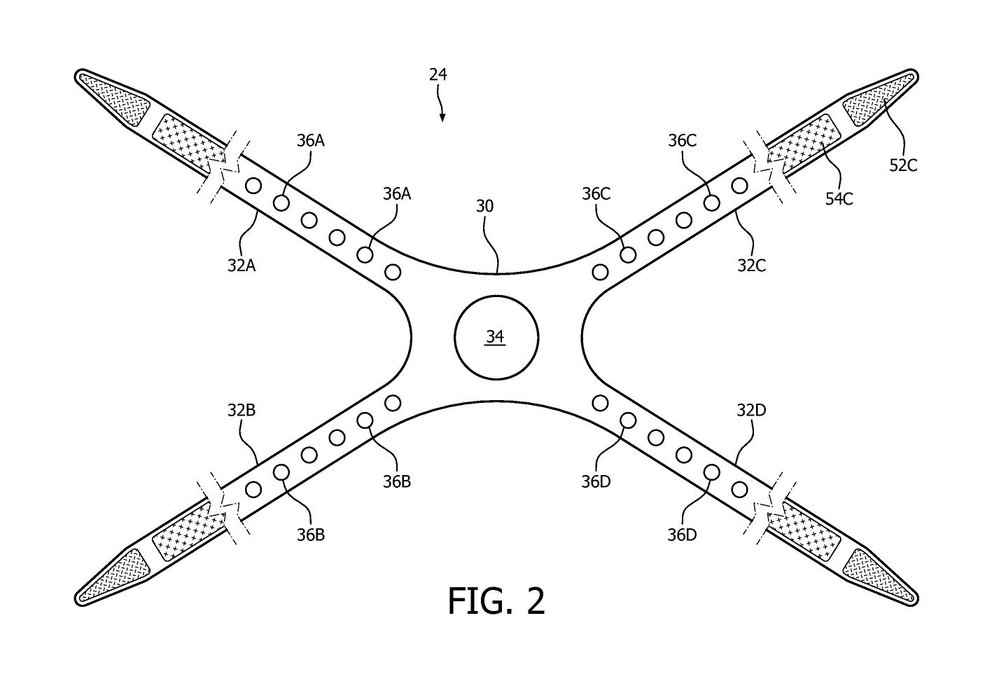

FIG. 2 is a top plan view of strap member 24 according to an exemplary embodiment of the present invention. Strap member 24 includes a central panel 30 and four arm members 32A, 32B, 32C, 32D, wherein arm members 32A, 32B extend from a first side of central panel 30 and arm members 32C, 32D extend from a second, opposite side of central panel 30. Central panel 30 and arm members 32A, 32B, 32C, 32D are, in the exemplary embodiment, part of a unitary structure made of a highly elastic material that is not a textile (i.e., a non-textile material). As used herein, the word “textile” means a material consisting of a network of interlaced or otherwise entangled natural or artificial fibers made by, for example and without limitation, weaving, knitting, spreading, crocheting, or bonding (e.g., by chemical, mechanical, heat or solvent treatment) the fibers to form the network, and may include, for example, and without limitation, woven and nonwoven fabric materials. In the exemplary embodiment, the highly elastic material of central panel 30 and arm members 32A, 32B, 32C, 32D has a tensile modulus of 1.5 Mpa to 10 Mpa and/or an elongation (without break) of 400% to 1100% (alternatively, 600% to 1100%), and may be, for example and without limitation, a low durometer elastomer (e.g., without limitation, natural rubber, neoprene, neoprene, silicone, or polyurethane).

In one particular embodiment, for low pressure (5 to 10 cmH₂O) applications (e.g., employing nasal pillows or nasal mask type interfaces), the highly elastic material of central panel 30 and arm members 32A, 32B, 32C, 32D has a tensile modulus of 3 Mpa to 5 Mpa and/or an elongation (without break) of 800% to 1000%. In another particular embodiment, for mid-high pressure (10 to 20 cmH₂O) applications, the highly elastic material of central panel 30 and arm members 32A, 32B, 32C, 32D has a tensile modulus of 5 Mpa to 9 Mpa and/or an elongation (without break) of 400% to 600%. In addition, in the exemplary embodiment, central panel 30 and arm members 32A, 32B, 32C, 32D each have a thickness of 1.5 mm to 5 mm.

As seen in FIG. 2, central panel 30 includes a central orifice 34, the purpose of which is described elsewhere herein. In addition, each arm member 32A, 32B, 32C, 32D includes a series of attachment orifices 36 (labeled 36A, 36B, 36C, 36D) that are spaced along a portion of the length of arm member 32A, 32B, 32C, 32D. In the exemplary embodiment, attachment orifices 36 are spaced evenly the portion of the length of arm member 32A, 32B, 32C, 32D (e.g., one every 1 cm along an arm having a length of 25 cm to 38 cm).

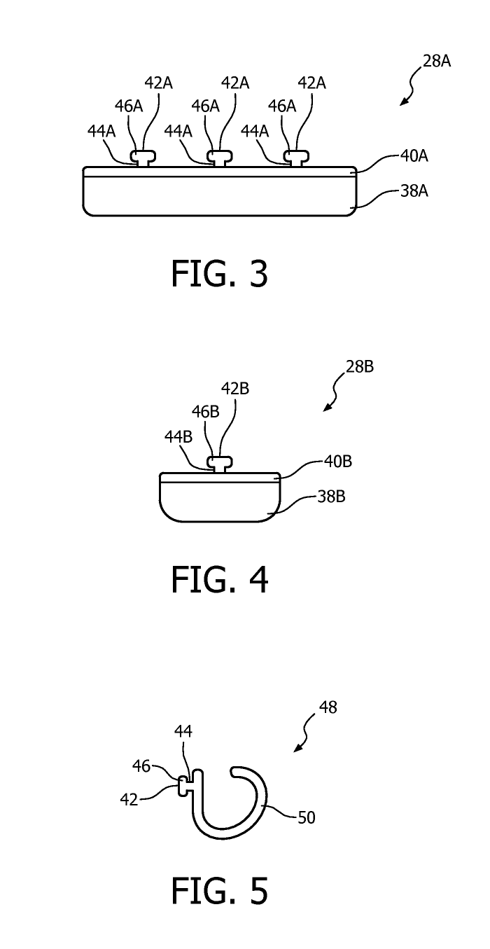

Attachment orifices 36 are provided to enable one or more suitably structured accessory elements, such as a pad member 28, to be selectively attached to any of arm members 32A, 32B, 32C, 32D at a desired location. In particular, FIG. 3 is a side elevational view of pad member 28A according to one non-limiting, exemplary embodiment. Pad member 28A includes a contact layer 38A coupled to a base/backing layer 40A. Contact layer 38A is structured to engage the face of the user of patient interface device 8, and may be made of a suitable, cushiony material such as, without limitation, a polyurethane based gel, a silicone based gel, or foam. In one specific embodiment, the material of contact layer 38 is tacky (at least at the outer surface) so as to prevent pad member 28A, and thus the rest of patient interface device 8 coupled to pad member 28A, from moving relative to the face of the user, thereby reducing the likelihood of abrasions being created. Base/backing layer 40A is made of a material that is of a higher durometer than contact layer 38A. Such material may include, without limitation, a thermoplastic material or a textile, such as a woven fabric material.

A number of button elements 42A (e.g., three) are provided on the surface of base/backing layer 40A opposite contact layer 38A. The spacing between each button element along base/backing layer 40A is the same as the spacing of attachment orifices 36 of arm members 32. Each button element 42A includes a post member 44A and an enlarged cap member 46A attached to post member 44A. Button element 42A may be made from any of a number of suitable materials, such as, without limitation, plastic (e.g., the same thermoplastic as base/backing layer 40A) or metal. Thus, pad member 28A is able to be selectively attached to any of the arm members 32A, 32B, 32C, 32D by inserting the button elements 42A through selected, appropriately spaced attachment orifices 36.

As will be appreciated, the elastic nature of arm members 32A, 32B, 32C, 32D will allow attachment orifices 36 to temporarily stretch to receive cap members 46A and then return to their original size around post members 44A. As will also be appreciated, any number of pad members 28A may be attached to any number of arm members 32A, 32B, 32C, 32D in this manner as desired. In addition, FIG. 4 is a side elevational view of pad member 28B according to another exemplary embodiment. Pad member 28B is similar to pad member 28A (like components are labeled with like reference numerals having a “B” designation rather than an “A” designation), except that it has a generally circular profile and is smaller than pad member 28A. Thus, pad member 28B is able to be selectively attached to any of the arm members 32A, 32B, 32C, 32D by inserting the button element 42B through a selected attachment orifice 36.

FIG. 5 is a side elevational view of an accessory in the form of a tube positioning/management clip 48 according to a further exemplary embodiment. Tube positioning/management clip 48 includes a looped holding portion 50 structured to receive and hold delivery conduit 6, and a button element 42 as described elsewhere herein. Thus, tube positioning/management clip 48 is able to be selectively attached to any of the arm members 32A, 32B, 32C, 32D by inserting the button element 42 through a selected attachment orifice 36 to enable the delivery conduit 6 to be held thereby in a desired position/location so as to no bother the user during therapy.

Referring again to FIG. 2, in the illustrated embodiment, the distal end of each of the arm members 32A, 32B, 32C, 32D is provided with a hook and loop fastening system (e.g., Velcro®) on the top surface thereof that comprises a hook portion 52A, 52B, 52C, 52D spaced from a loop portion 54A, 54B, 54C, 54D. These hook and loop fastening systems enable strap member 24 (having one or more accessories coupled thereto as described herein) to be adjustably coupled to back piece 26 by inserting each distal end of each arm member 32A, 32B, 32C, 32D through a respective looped connector 56 (FIG. 1) provided on back piece 26 and then bending the distal end back on itself at a desire location to secure the hook portion 52 to the loop portion 54. The hook and loop fastening system as just described is not meant to be limiting, and it will be understood that other types of fastening systems are also contemplated within the scope of the present invention.

Thus, in order to don patient interface device 8, the user will typically first position cushion assembly 12 within the rear of central panel 30 of strap member 24 (having one or more accessories coupled thereto as described herein) in a manner wherein a portion of faceplate member 14 and elbow conduit 10 are inserted through central orifice 34 as seen in FIG. 1. The user will then place headgear assembly 16 onto his or her head and adjust each arm member 32A, 32B, 32C, 32D as just described until an appropriate fit and seal are achieved.

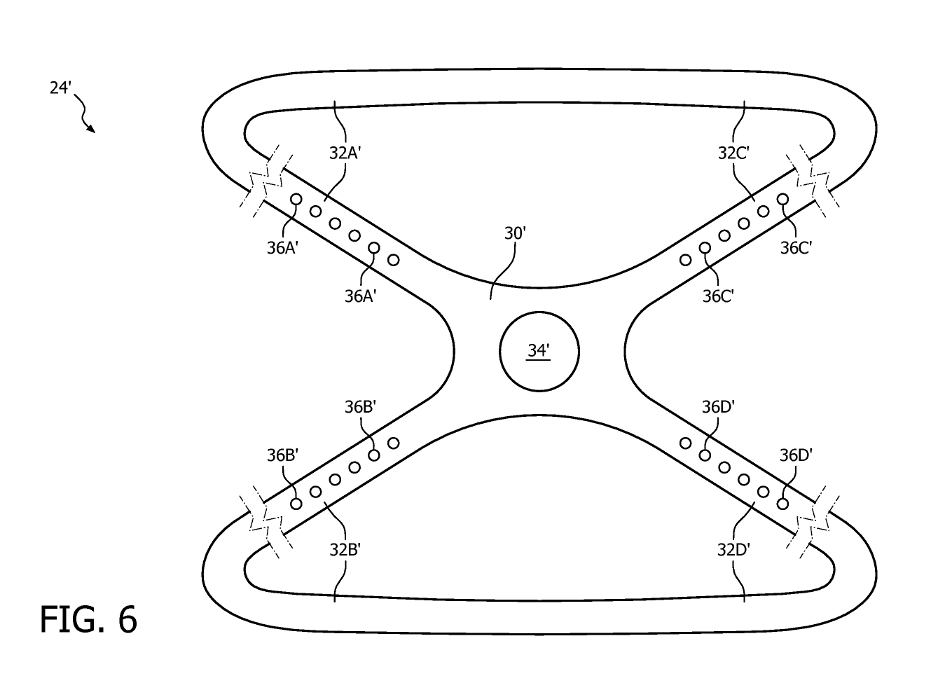

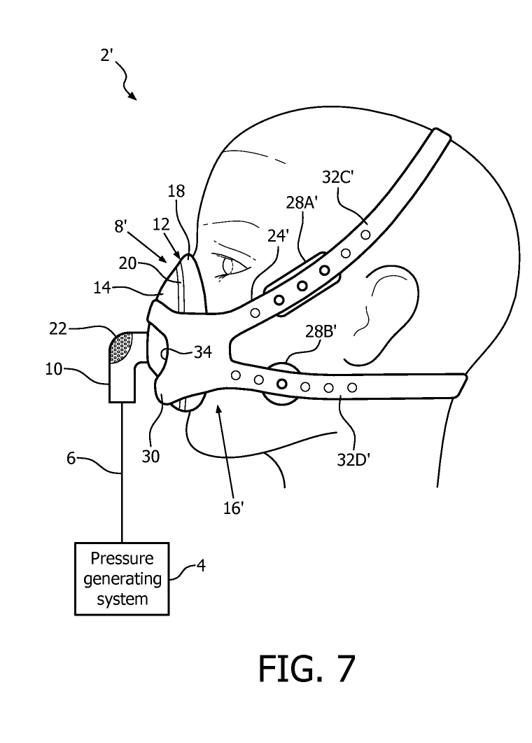

In an alternative embodiment, rather than the distal end of each arm member 32A, 32B, 32C, 32D being provided with a hook and loop fastening system as just described, arm member 32A may be attached to arm member 32C and arm member 32B may be attached to arm member 32D as shown in FIG. 6 (which is a top plan view of an alternative strap member 24′ showing alternative arm members 32A′, 32B′, 32C′, 32D′ thus configured). In such an implementation, patient interface device 8′ forming a part of an alternative system 2′, shown in FIG. 7, may be donned by simply placing arm members 32A′, 32B′, 32C′, 32D′ over the user's head. Adjustment in this embodiment is provided automatically by the elastic properties of alternative strap member 24′, which may be made of the same materials that were described in connection with strap member 24.

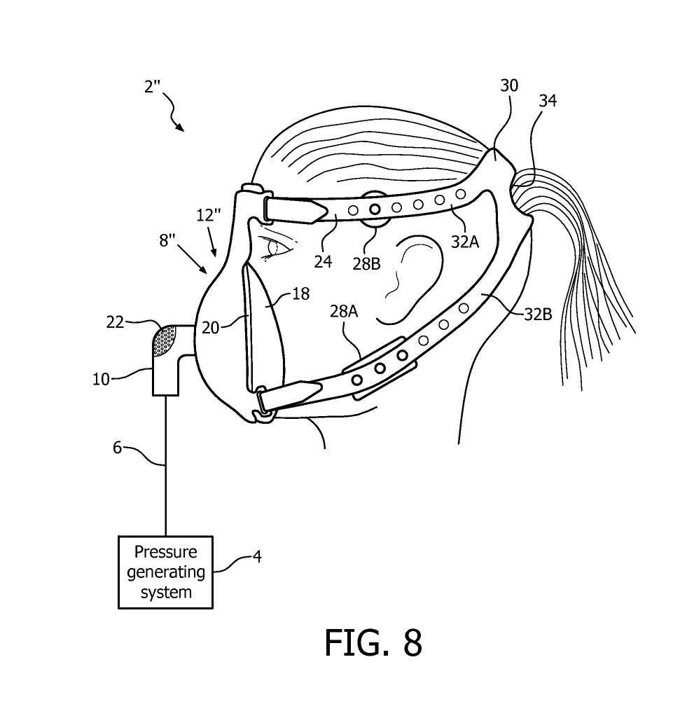

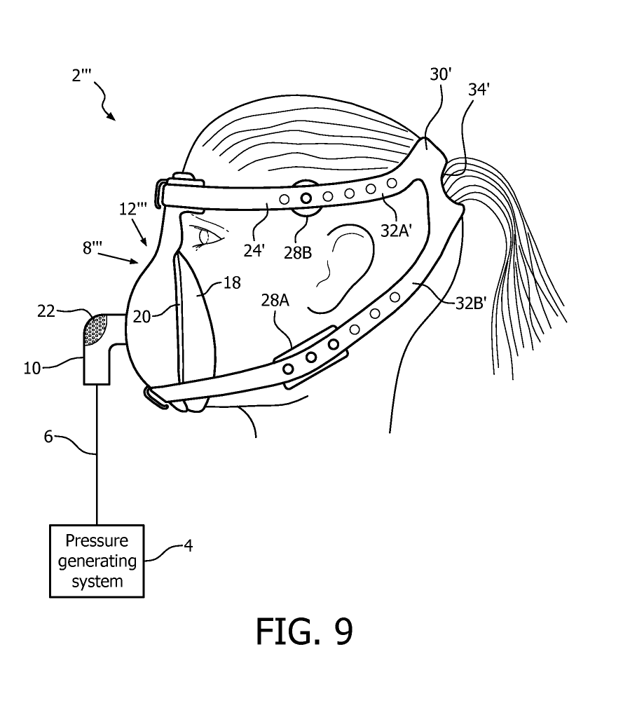

Moreover, according to a further aspect of the invention, strap member 24 and alternative strap member 24′ are reversible as shown in FIGS. 8 and 9, respectively, wherein they are used to implement further alternative systems 2″ (FIG. 8) and 2′″ (FIG. 9) employing alternative patient interface devices 8″ and 8′″. By reversible, it is meant that central panel 30 and central panel 30′, rather than being used as a holder and stabilizer for cushion assembly 12 as seen in FIGS. 1 and 7, are instead deployed at the back of the user's head as a stabilizer and, for example, a pony tail holder. In addition, in the embodiments of FIGS. 8 and 9, alternative cushion assemblies 12″ and 12′″ are employed that each have a frame member having a faceplate portion and a forehead support portion.

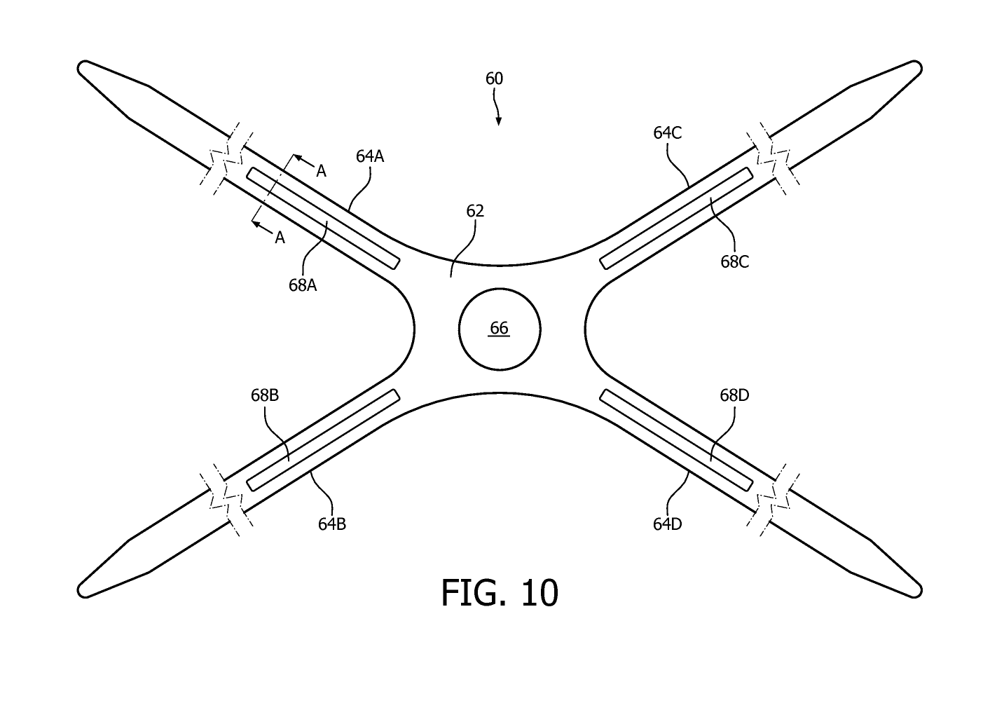

FIG. 10 is a bottom plan view of a strap member 60 according to another, alternative exemplary embodiment of the present invention that may be used in place of strap member 24. Strap member 60 includes a central panel 62 and four arm members 64A, 64B, 64C, 64D, wherein arm members 64A, 64B extend from a first side of central panel 62 and arm members 64C, 64D extend from a second, opposite side of central panel 62. Central panel 62 and arm members 64A, 64B, 64C, 64D are, in the exemplary embodiment, part of a unitary structure made of a highly elastic material that is not a textile (i.e., a non-textile material). In the exemplary embodiment, the highly elastic material of central panel 62 and the portion of arm members 64A, 64B, 64C, 64D not forming channels 68 has a tensile modulus of 1.5 Mpa to 10 Mpa and/or an elongation (without break) of 400% to 1100% (alternatively, 600% to 1100%), and may be, for example and without limitation, a low durometer elastomer (e.g., without limitation, natural rubber, neoprene, silicone, or polyurethane).

In one particular embodiment, for low pressure (5 to 10 cmH₂O) applications (e.g., employing nasal pillows or nasal mask type interfaces), the highly elastic material of central panel 62 and the portion of arm members 64A, 64B, 64C, 64D not forming channels 68 has a tensile modulus of 3 Mpa to 5 Mpa and/or an elongation (without break) of 800% to 1000%. In another particular embodiment, for mid-high pressure (10 to 20 cmH₂O) applications, the highly elastic material of central panel 62 and the portion of arm members 64A, 64B, 64C, 64D not forming channels 68 has a tensile modulus of 5 Mpa to 9 Mpa and/or an elongation (without break) of 400% to 600%. Furthermore, the portion of arm members 64A, 64B, 64C, 64D forming channels 68 (i.e., the wall portion within 5-8 mm, or approximately ⅓ of overall strap width, of the void of the channels 68) has a higher tensile modulus of 8 Mpa to 10 Mpa and a lower elongation (without break) of less than or equal to 650%. As will be appreciated, these portions of differing tensile modulus may be made using a two step molding process. In addition, in the exemplary embodiment, central panel 62 and the portion of arm members 64A, 64B, 64C, 64D not forming channels 68 each have a thickness of 1.5 mm to 3 mm, and the portion of arm members 64A, 64B, 64C, 64D forming channels 68 have a thickness of 2.5 mm to 5 mm.

As seen in FIG. 10, central panel 62 includes a central orifice 66. In addition, each arm member 64A, 64B, 64C, 64D includes an attachment channel 68 (labeled 68A, 68B, 68C, 68D) provided in the bottom surface thereof that extends along a portion of the length thereof. FIG. 11 is a cross-sectional view taken along lines A-A of FIG. 10 which shows attachment channel 68A. As will be appreciated, attachment channels 68B, 68C, and 68D will have a similar structure. In addition, the top surface of the distal end of each arm member 64A, 64B, 64C, 64D includes a hook and loop fastening system (not shown) to enable it to be coupled to adjustably coupled to the head of the user as described elsewhere herein.

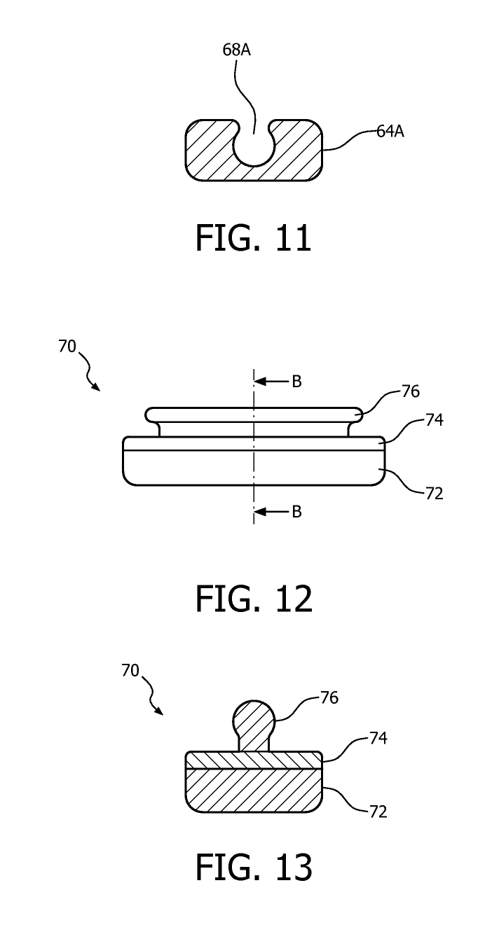

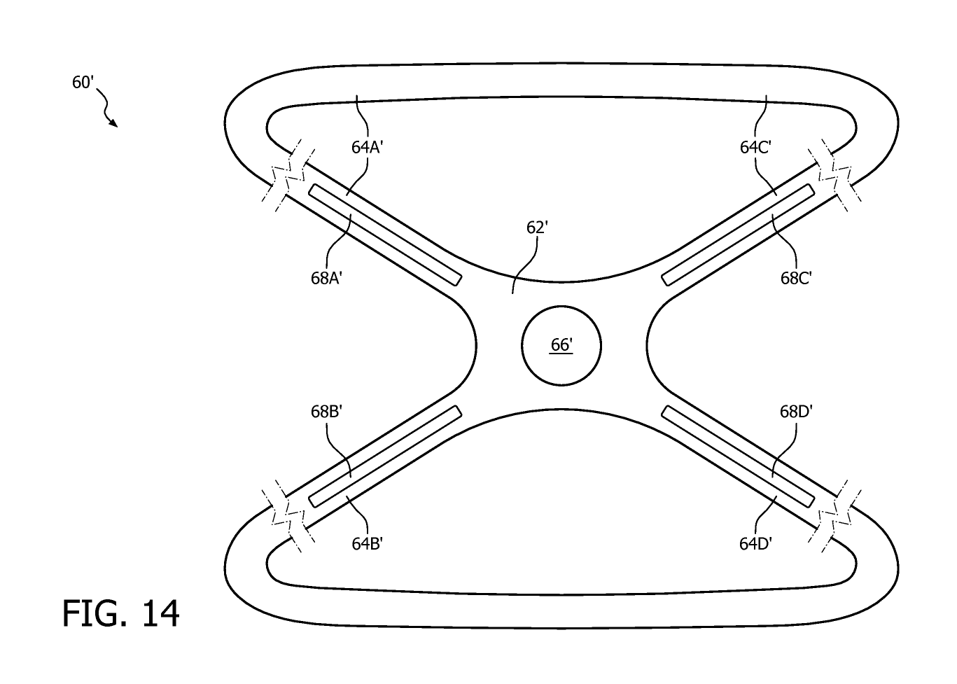

Attachment channels 68 are provided to enable one or more suitably structured accessory elements, such as a pad member 70 shown in FIGS. 12 and 13, to be selectively attached to any of arm members 64A, 64B, 64C, 64D. FIG. 12 is a side elevational view of pad member 70 and FIG. 13 is a cross-sectional view of pad member 70 taken along lines B-B of FIG. 12 according to one exemplary embodiment. Pad member 70 includes a contact layer 72 coupled to a base/backing layer 74, which are similar in structure, function and material to contact layer 38A base/backing layer 40A described elsewhere herein. In addition, a mating pin member 76 is provided on the surface of base/backing layer 74 opposite contact layer 72. Mating pin member 76 is sized to be securely received and held within any of the flexible attachment channels 68B, 68C, and 68D.

In the exemplary embodiment, the attachment channels 68 are longer than the mating pin member 76. Thus, pad member 70 is able to be selectively attached to any of the arm members 64A, 64B, 64C, 64D at a desired location by inserting mating pin member 76 into an attachment channel at the desired location. As will be appreciated, the elastic nature of arm members 64A, 64B, 64C, 64D will allow attachment channels 68 to temporarily expand to receive mating pin member 76 and then retract to hold mating pin member 76. As will also be appreciated, any number of pad members 70 (or alternative accessories (e.g., a circular pad member or a tube position/management clip) having a suitable mating pin member) may be attached to any number of arm members 64A, 64B, 64C, 64D in this manner as desired.

In an alternative embodiment, rather than the distal end of each arm member 64A, 64B, 64C, 64D being provided with a hook and loop fastening system as just described, arm member 64A may be attached to arm member 64C and arm member 64B may be attached to arm member 64D as shown in FIG. 14 (which is a bottom plan view of an alternative strap member 60′ showing alternative arm members 64A′, 64B′, 64C′, 64D′ thus configured). In such an implementation, a patient interface device employing strap member 60′ may be donned by simply placing arm members 64A′, 64B′, 64C′, 64D′ over the user's head. Adjustment in this embodiment is provided automatically by the elastic properties of alternative strap member 60′. Furthermore, according to a further aspect of the invention, strap member 60 and alternative strap member 60′ are reversible as described elsewhere herein.

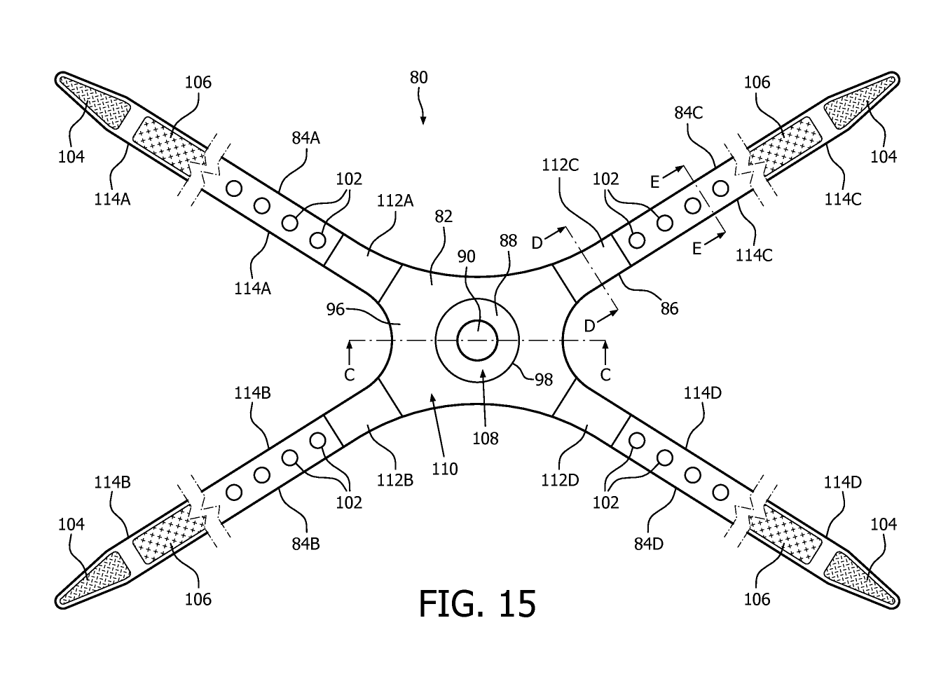

FIG. 15 is a top plan view of a strap member 80 according to still another, alternative exemplary embodiment of the present invention that may be used in place of strap member 24. Strap member 80 includes a central panel 82 and four arm members 84A, 84B, 84C, 84D, wherein arm members 84A, 84B extend from a first side of central panel 82 and arm members 84C, 84D extend from a second, opposite side of central panel 82.

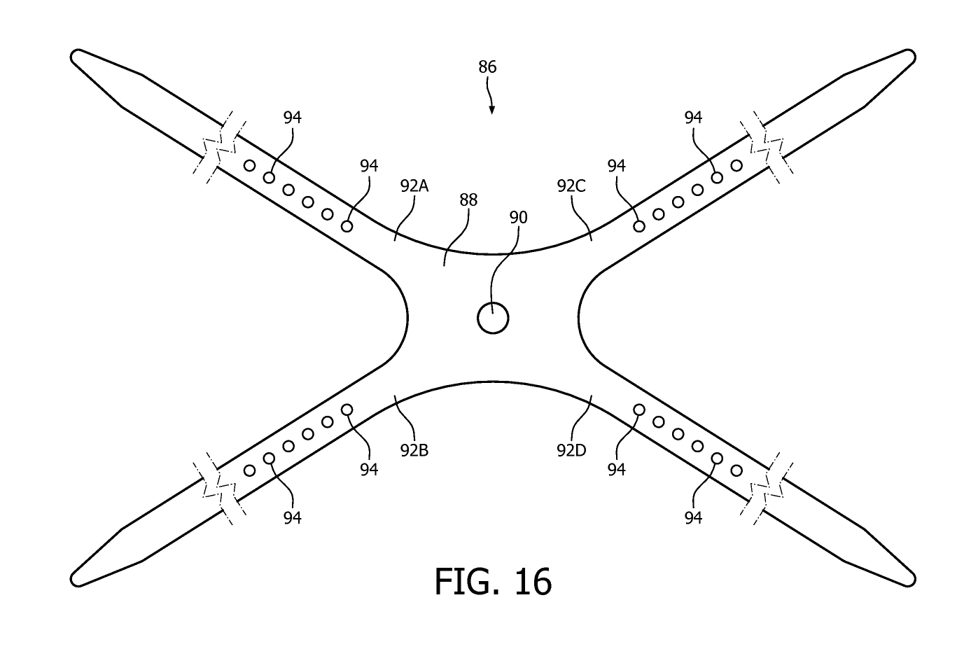

Strap member 80 is of the present embodiment is formed as a reinforced composite structure. More specifically, strap member 80 comprises a base member 86, shown in top plan view in FIG. 16, having a central panel portion 88 defining an orifice 90, and arm members 92A, 92B, 92C, 92D having orifices 94. In the exemplary embodiment, base member 86 is a unitary structure made of a highly elastic material. In the exemplary embodiment, the highly elastic material of base member 86 has a tensile modulus of 1.5 Mpa to 10 Mpa and/or an elongation (without break) of 400% to 1100% (alternatively, 400% to 1100%), and may be, for example and without limitation, a low durometer elastomer, or a flexible, elastic textile. In one embodiment, if a flexible textile is employed, the weave thereof should be such that it will stretch only along the longitudinal axes of the arm members 92A, 92B, 92C, 92D.

In one particular embodiment, for low pressure (5 to 10 cmH₂O) applications (e.g., employing nasal pillows or nasal mask type interfaces), the highly elastic material of base member 86 has a tensile modulus of 3 Mpa to 5 Mpa and/or an elongation (without break) of 800% to 1000%. In another particular embodiment, for mid-high pressure (10 to 20 cmH₂O) applications, the highly elastic material of base member 86 has a tensile modulus of 5 Mpa to 9 Mpa and/or an elongation (without break) of 400% to 600%. In addition, in the exemplary embodiment, base member 86 has a thickness of 1.5 mm to 3 mm.

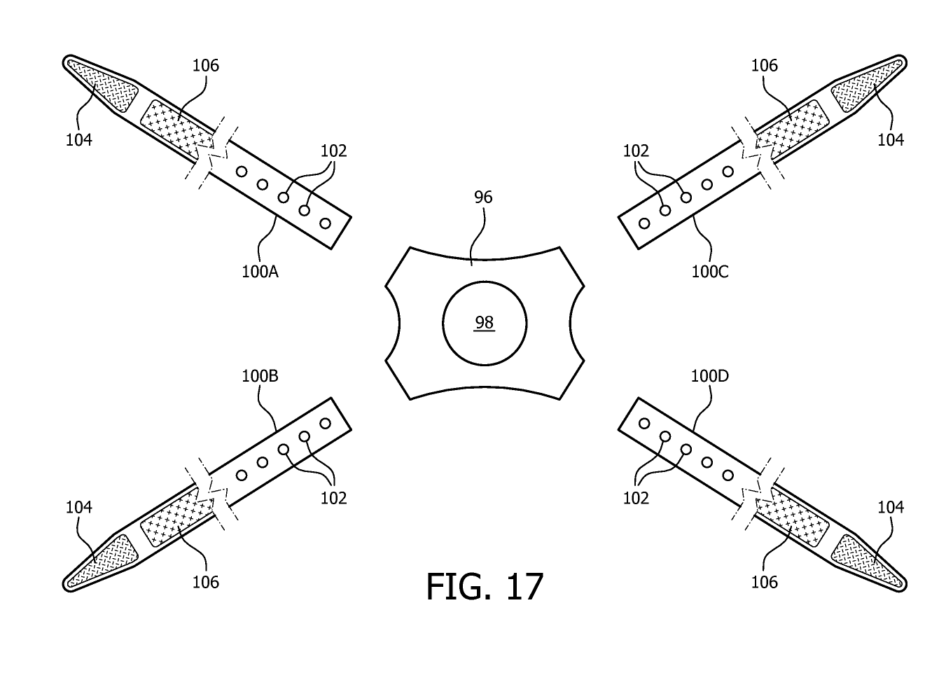

In addition, strap member 80 also includes a plurality of reinforcing portions that are provided on top of and attached to base member 86. In particular, as seen in FIG. 17, such reinforcing portions include a central reinforcing portion 96 having a central orifice 98, and arm reinforcing portions 100A, 100B, 100C, 100D having orifices 102. The distal ends of arm reinforcing portions 100A, 100B, 100C, 100D are provided with a hook and loop fastening system comprising a hook portion 104 and a loop portion 106 as described elsewhere herein. In the exemplary embodiment, central reinforcing portion 96 and arm reinforcing portions 100A, 100B, 100C, 100D are made of an inelastic material having a tensile modulus greater than or equal to 15 Mpa and/or an elongation (without break) of less than or equal to 60%, and may include a textile material, such as a woven fabric, or a thermoplastic material. Also, the thickness of each of central reinforcing portion 96 and arm reinforcing portions 100A, 100B, 100C, 100D may be 0.2 mm to 1.5 mm and could be layered as necessary to create a structure up to 5 to 6 mm thick in total.

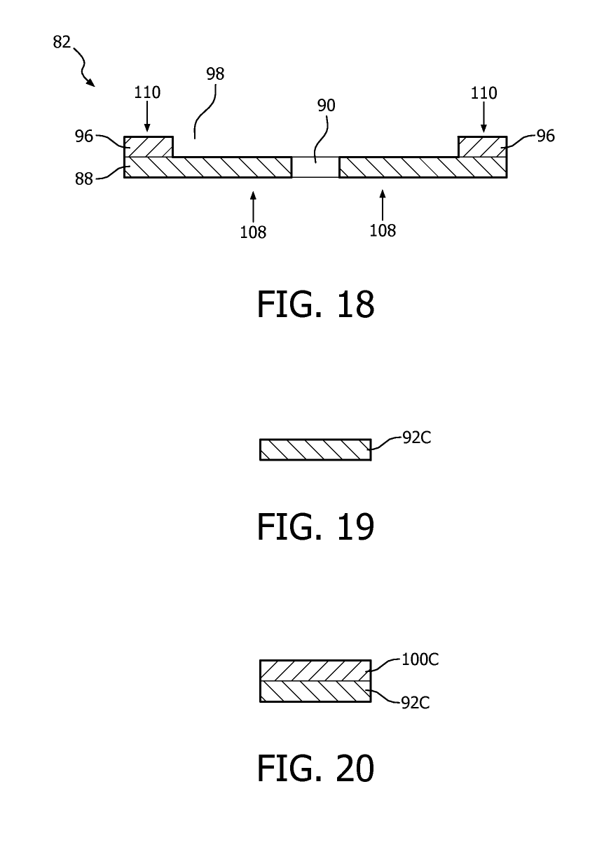

Thus, when formed, as seen in FIG. 15, strap member 80 will include certain reinforced portions (having both the elastic and inelastic materials) and certain non-reinforced portions (having only the elastic material). In particular, central panel 82 has a central non-reinforced portion 108 surrounded by an outer reinforced portion 110. These sections are shown in FIG. 18, which is a cross-sectional view of strap member 80 taken along lines C-C in FIG. 15. Also, each arm member 84 has an inner, proximal non-reinforced portion 112 located immediately adjacent central panel 82, and an outer, distal reinforced portion 114 spaced from central panel 82 by non-reinforced portion 112. These sections are shown in FIGS. 19 and 20, which are cross-sectional views of strap member 80 taken along lines D-D and E-E, respectively, in FIG. 15.

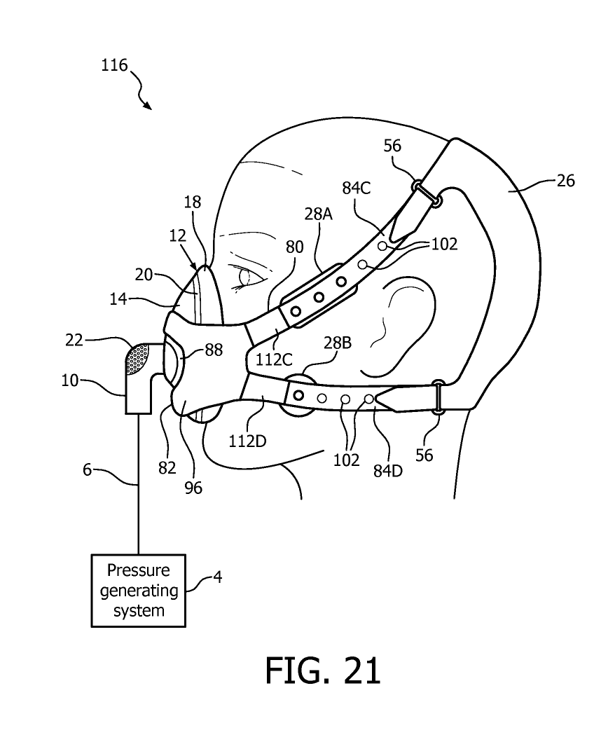

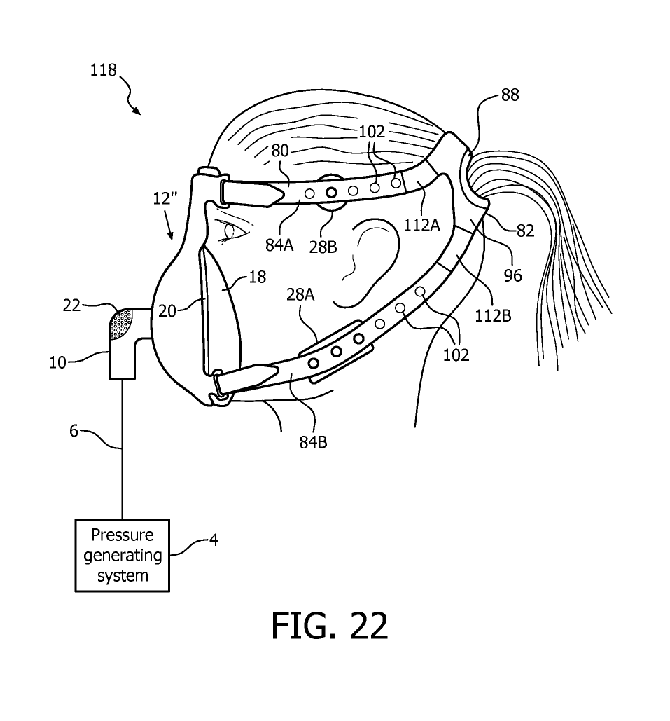

Strap member 80 may thus be used to implement a system 116 and a system 118, shown in FIGS. 21 and 22, respectively, which are similar to system 2 of FIG. 1 and system 2″ of FIG. 8 (like components are labeled with like reference numerals).

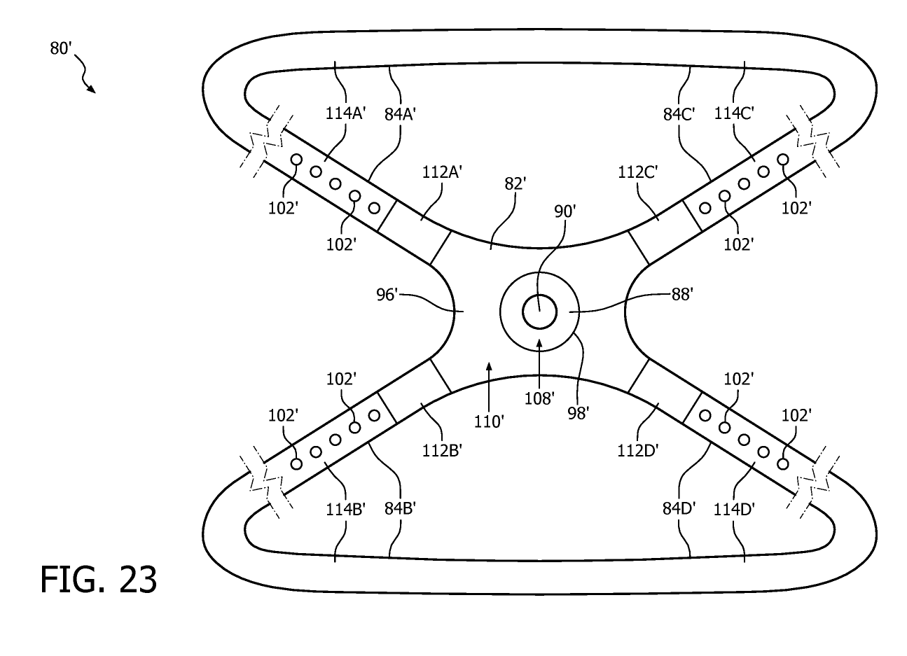

In an alternative embodiment, rather than the distal end of each arm member 84A, 84B, 84C, 84D being provided with a hook and loop fastening system as described above, arm member 84A may be attached to arm member 84C and arm member 84B may be attached to arm member 84D as shown in FIG. 23 (which is a top plan view of an alternative strap member 80′ showing alternative arm members 84A′, 84B′, 84C′, 84D′ thus configured; other similar components are denoted by like reference numerals followed by a “′” symbol). In such an implementation, a patient interface device employing strap member 80′ may be donned in system 2′ or system 2′″ in place of strap member 24′ by simply placing arm members 84A′, 84B′, 84C′, 84D′ over the user's head. Adjustment in this embodiment is provided automatically by the elastic properties of non-reinforced portions 112A′ through 112D′.

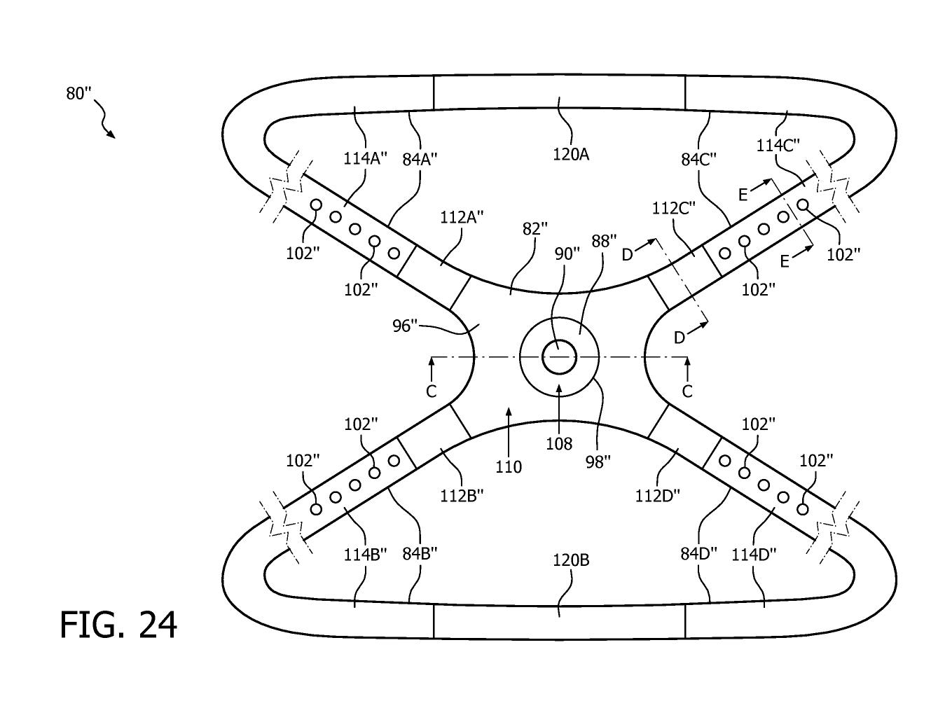

FIG. 24 is a top plan view of another alternative strap member 80″ showing alternative arm members 84A″, 84B″, 84C″, 84D″, wherein arm member 84A″ is attached to arm member 84C″ and arm member 84B″ may is attached to arm member 84D″ (other similar components are denoted by like reference numerals followed by a “″” symbol). As seen in FIG. 24, strap member 80″ includes an additional non-reinforced section 120A at the point where arm member 84A″ is attached to arm member 84C″ and an additional non-reinforced section 120B at the point where arm member 84B″ is attached to arm member 84D″. In such an implementation, a patient interface device employing strap member 80″ may be donned in system 2′ or system 2′ in place of strap member 24′, wherein additional non-reinforced sections 120A and 120B will provide additional elastic sections to facilitate self-adjustment.

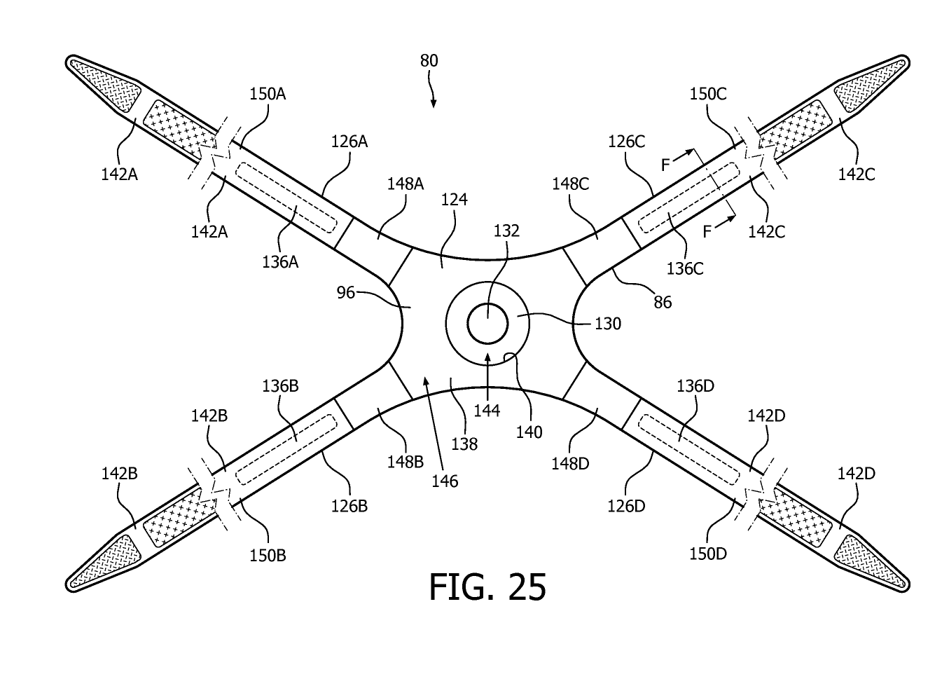

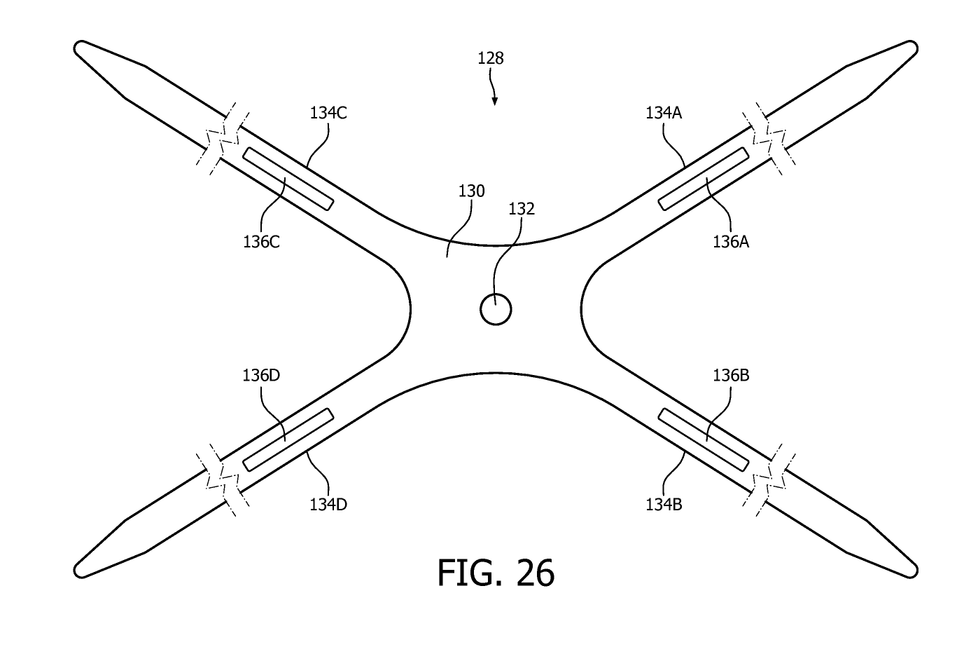

FIG. 25 is a top plan view of a strap member 122 according to still another, alternative exemplary embodiment of the present invention. Strap member 122 is similar to strap member 80 in that it includes a central panel 124 and four arm members 126A, 126B, 126C, 126D. In addition, strap member 122 is formed as a reinforced composite structure. Strap member 122 thus includes a base member 128, shown in bottom plan view in FIG. 26, having a central panel portion 130 defining an orifice 132, and arm members 134A, 134B, 134C, 134D. Arm members 134A, 134B, 134C, 134D each include an attachment channel 136 that is similar to attachment channels 68 (FIG. 10). In the exemplary embodiment, base member 128 is a unitary structure made of a highly elastic material. In the exemplary embodiment, the highly elastic material in the portion of base member 128 not forming channels 136 has a tensile modulus of 1.5 Mpa to 10 Mpa and/or an elongation (without break) of 400% to 1100% (alternatively, 400% to 1100%), and may be, for example and without limitation, a low durometer elastomer (e.g., without limitation, natural rubber, neoprene, silicone, or polyurethane), or a flexible, elastic textile. In one particular embodiment, for low pressure (5 to 10 cmH₂O) applications (e.g., employing nasal pillows or nasal mask type interfaces), the highly elastic material of base member 128 not forming channels 136 has a tensile modulus of 3 Mpa to 5 Mpa and/or an elongation (without break) of 800% to 1000%.

In another particular embodiment, for mid-high pressure (10 to 20 cmH₂O) applications, the highly elastic material of base member 128 not forming channels 136 has a tensile modulus of 5 Mpa to 9 Mpa and/or an elongation (without break) of 400% to 600%. Furthermore, the portion of base member 128 forming channels 136 (i.e., the wall portion within 5-8 mm, or approximately ⅓ of overall strap width, of the void of the channels 136) has a higher tensile modulus of 8 Mpa to 10 Mpa and a lower elongation (without break) of less than or equal to 650%. As will be appreciated, these portions of differing tensile modulus may be made using a two step molding process. In addition, in the exemplary embodiment, base member 128 has a thickness of 1.5 mm to 3 mm.

Strap member 122 is formed by attaching a plurality of reinforcing portions to the top of base member 126. In particular, as seen in FIG. 25, a central reinforcing portion 138 having a central orifice 140, and arm reinforcing portions 142A, 142B, 142C, 142D are attached to the top of base member 126.

Thus, when formed, as seen in FIG. 25, strap member 122 will include certain reinforced portions (having both the elastic and inelastic materials) and certain non-reinforced portions (having only the elastic material). In particular, central panel 124 has a central non-reinforced portion 144 surrounded by an outer reinforced portion 146. Also, each arm member 126 has an inner, proximal non-reinforced portion 148 located immediately adjacent central panel 124, and an outer, distal reinforced portion 150 spaced from central panel 124 by non-reinforced portion 148.

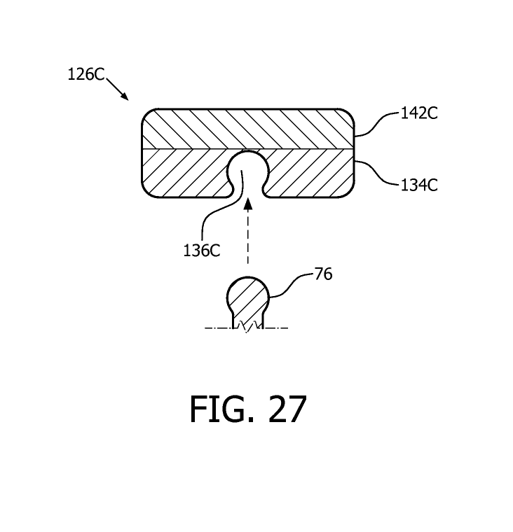

In addition, the provision of attachment channels 136 allows suitably structured accessories (e.g., pad 70 with mating pin member 76) to be selectively attached to arm members 126A, 126B, 126C, 126D in the manner described elsewhere herein. This is illustrated in FIG. 27, which includes a cross-section of arm member 126C taken along lines F-F in FIG. 25.

In another alternative embodiment, rather than the distal end of each arm member 126A, 126B, 126C, 126D being provided with a hook and loop fastening system as described above, arm member 126A may be attached to arm member 126C and arm member 126B may be attached to arm member 126D in a manner similar to that shown in FIGS. 23 and 24 and described herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A headgear assembly for a patient interface device structured to deliver a flow of breathing gas to a patient, comprising:
a strap member having a central panel and at least a first arm member extending from a first side of the central panel and a second arm member extending from a second side of the central panel opposite the first side, the central panel, the first arm member and the second arm member being made of an elastic non-textile material having at least one of a tensile modulus of 1.5 Mpa to 10 Mpa and an elongation without break of 400% to 1100%, the first arm member and the second arm member each having at least one first attachment feature;
at least one accessory member having at least one second attachment feature, the at least one second attachment feature is selectively mateable with the at least one first attachment feature to allow the at least one accessory member to be selectively attached to and positioned along either the first arm member or the second arm member at a plurality of different locations.

2. The headgear assembly according to claim 1, wherein the elastic non-textile material has both a tensile modulus of 1.5 Mpa to 10 Mpa and an elongation without break of 400% to 1100%.

3. The headgear assembly according to claim 1, wherein the elastic non-textile material has both a tensile modulus of 1.5 Mpa to 10 Mpa and an elongation without break of 600% to 1100%.

4. The headgear assembly according to claim 1, wherein the elastic non-textile material has both a tensile modulus of 3 Mpa to 5 Mpa and an elongation without break of 800% to 1000%.

5. The headgear assembly according to claim 1, wherein the elastic non-textile material has both a tensile modulus of 5 Mpa to 9 Mpa and an elongation without break of 400% to 600%.

6. The headgear assembly according to claim 1, wherein in the first arm member and the second arm member the at least one first attachment feature comprises a plurality of orifices provided along a portion of a length of the first arm member and the second arm member, and wherein the at least one second attachment feature comprises at least one post member.

7. The headgear assembly according to claim 1, wherein in the first arm member and the second arm member the at least one first attachment feature comprises a channel provided along a portion of a length of the first arm member and the second arm member, and wherein the at least one second attachment feature comprises a mating pin member structured to be received within the channel.

8. The headgear assembly according to claim 7, wherein a portion of the first arm member and a portion the second arm member forming the at least one first attachment feature is made of a material having a tensile modulus of 8 Mpa to 10 Mpa and an elongation without break of less than or equal to 650%.

9. The headgear assembly according to claim 1, wherein the at least one accessory member is a pad member or a clip member.

10. The headgear assembly according to claim 1, wherein the central panel includes a central orifice.

11. The headgear assembly according to claim 10, wherein the strap member is reversible such that the central panel is structured to receive and hold a cushion assembly of the patient interface device or be positioned on a back of the patient's head when the patent interface device is donned by the patient.

12. The headgear assembly according to claim 1, wherein the strap member further includes a third arm member extending from the first side of the central panel and a fourth arm member extending from the second side of the central panel, the third arm member and the fourth arm member being made of the elastic non-textile material and each having the at least one first attachment feature.

13. The headgear assembly according to claim 1, wherein the strap member is a unitary structure wherein the first arm member is directly coupled to the second arm member.

14. A patient interface device including a cushion assembly and a headgear assembly according to claim 1 coupled to the cushion assembly.

15. A system for delivering a flow of breathing gas to a patient, comprising a pressure generating system structured to generate the flow of breathing gas and a patient interface device according to claim 14 fluidly coupled to the pressure generating system.

16. A headgear assembly for a patient interface device structured to deliver a flow of breathing gas to a patient, comprising:
a strap member including: (i) a base member made of an elastic material having at least one of a tensile modulus of 1.5 Mpa to 10 Mpa and an elongation without break of 400% to 1100%, wherein the base member includes a central panel portion (88, 130) and at least a first base arm member extending from a first side of the central panel portion and a second base arm member extending from a second, opposite side of the central panel portion, wherein the first base arm member and the second base arm member each have at least one first attachment feature, (ii) a central reinforcing portion (96) coupled to and covering only a portion of the central panel portion, (iii) a first arm reinforcing portion coupled to and covering only a portion of the first base arm member, and (iv) a second arm reinforcing portion coupled to and covering only a portion of the second base arm member, wherein the central reinforcing portion, the first arm reinforcing portion and the second arm reinforcing portion are made of an inelastic material having at least one of a tensile modulus greater than or equal to 15 Mpa and an elongation without break of less than or equal to 60%; and
at least one accessory member having at least one second attachment feature, wherein the at least one second attachment feature is selectively mateable with the at least one first attachment feature to allow the at least one accessory member to be selectively attached to and positioned along either the first base arm member or the second base arm member at a plurality of different locations.

17. The headgear assembly according to claim 16, wherein the elastic material has both a tensile modulus of 1.5 Mpa to 10 Mpa and an elongation without break of 400% to 1100%.

18. The headgear assembly according to claim 16, wherein the elastic material has both a tensile modulus of 1.5 Mpa to 10 Mpa and an elongation without break of 600% to 1100%.

19. The headgear assembly according to claim 16, wherein the elastic material has both a tensile modulus of 3 Mpa to 5 Mpa and an elongation without break of 800% to 1000%.

20. The headgear assembly according to claim 16, wherein the elastic material has both a tensile modulus of 5 Mpa to 9 Mpa and an elongation without break of 400% to 600%.

21. The headgear assembly according to claim 16, wherein the inelastic material has a tensile modulus greater than or equal to 15 Mpa and an elongation without break of less than or equal to 60%.

22. The headgear assembly according to claim 16, wherein the central panel portion includes a first central orifice and the central reinforcing portion includes a second central orifice, wherein the first central orifice is smaller than and positioned within the second central orifice.

23. The headgear assembly according to claim 16, wherein a first section of the first base arm member immediately adjacent the central reinforcing portion is not covered by the first arm reinforcing portion and a first section of the second base arm member immediately adjacent the central reinforcing portion is not covered by the second arm reinforcing portion.

24. The headgear assembly according to claim 16, wherein in the first base arm member and the second base arm member the at least one first attachment feature comprises a plurality of orifices provided along a portion of a length of the first base arm member and the second base arm member, and wherein the at least one second attachment feature comprises at least one post member.

25. The headgear assembly according to claim 16, wherein in the first base arm member and the second base arm member the at least one first attachment feature comprises a channel provided along a portion of a length of the first base arm member and the second base arm member, and wherein the at least one second attachment feature comprises a mating pin member structured to be received within the channel.

26. The headgear assembly according to claim 16, wherein the base member is a unitary structure wherein the first base arm member is directly coupled to the second base arm member.

27-28. (canceled)
